SEQUENCE LISTING (7 sequences)

SEQ ID NO: 1 - DNA, 3780 bp, Virus cDNA
  cDNA (1)..(3780)
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgcgcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatgg

SEQ ID NO: 2 - DNA, 26 bp, Artificial Sequence
  PCR Primer for Spike Gene N-terminal region amplification
ggtctagagttgtggtttcaagtgat

SEQ ID NO: 3 - DNA, 28 bp, Artificial Sequence
  PCR Primer for Spike Gene Middle region amplification
tttctagaccatgggttgtgtccttgct

SEQ ID NO: 4 - DNA, 29 bp, Artificial Sequence
  PCR Primer for Spike Gene C-treminal region amplification
tttctagaccatggcatataggttcaatg

SEQ ID NO: 5 - DNA, 24 bp, Artificial Sequence
  PCR Primer for Spike Gene N-treminal region amplification
taggtaccaatgccagtagtggtg

SEQ ID NO: 6 - DNA, 22 bp, Artificial Sequence
  PCR Primer for Spike Gene Middle region amplification
ttggtacctccgcctcgacttt

SEQ ID NO: 7 - DNA, 26 bp, Artificial Sequence
  PCR Primer for Spike Gene C-treminal region amplification
ccggtaccataagttcgtttatgtgt

(19) United States
(12) Patent Application Publication
(10) Pub. No.: US 2008/0267992 A1
(43) Pub. Date: Oct. 30, 2008

(54) Sars Virus Vaccine with Adenovirus Carrier and Preparation Method Thereof, and Use of Sars Virus S Gene for Preparation of Vaccine

(75) Inventors: Wenlin Huang, Guangzhou (CN); Yixin Zeng, Guangzhou (CN); Jian Wang, Guangzhou (CN); Ranyi Liu, Guangzhou (CN); Jialing Huang, Guangzhou (CN); Bijun Huang, Guangzhou (CN); Kun Lai, Guangzhou (CN); Lizhi Wu, Guangzhou (CN); Zhihui Liang, Guangzhou (CN); Miaola Ke, Guangzhou (CN); Xiuju Wu, Guangzhou (CN)

Correspondence Address: OSHA LIANG L.L.P., 1221 MCKINNEY STREET, SUITE 2800, HOUSTON, TX, 77010, US

(73) Assignee: Cancer Center, Sun Yat-Sun University, Guangzhou Shi, Guangdong (CN)

(21) Appl. No.: 11/628,518

(22) PCT Filed: Jun. 4, 2004
(86) PCT No.: PCT/CN04/00505

§ 371 Date: Dec. 4, 2006

(51) Int. Cl. A61K 39/00; C12N 15/87; A61P 37/00

(52) U.S. Cl. 424/202.1; 435/456; 424/204.1; 424/233.1

(57) ABSTRACT

The present invention relates to the field of biological engineering technology, specifically, to a SARS virus vaccine with adenovirus carrier, preparation method thereof and use of SARS virus S gene for preparation of severe acute respiratory syndrome (SARS) virus vaccine.

### FIELD OF INVENTION

This invention relates to genetic engineering and the use of adenovirus vector in the preparation of SARS vaccines and the use of coronavirus S gene in the preparation of vaccines. Particularly, this invention relates to the use of the S gene of SARA-related coronavirus in vaccine preparation for preventing SARS.

### BACKGROUND ARTS

Atypical pneumonia outbreak in Guangdong province in China in 2003 exhibited acute onset, strong infectivity and resistance to antibiotics. Currently, this disease has spread to more than 30 countries and areas. The WHO has designated this disease as severe acute respiratory syndrome (SARS).

At present, scientists from many countries have isolated a new type of coronavirus from serum samples of different patients. The results from gene sequence analyses of the virus indicate that this virus has 50%˜60% homology with the known coronavirus. The WHO has declared that the pathogen causing SARS is a variant of coronavirus.

Coronavirus is a non-segmented, positive-sense RNA virus. Its genome comprises nearly 30 kb, and can transmit between humans and animals, primarily infecting their respiratory systems. The coronavirus particle has an inner nucleus and a capsule, and includes four structural proteins, spike (S), membrane (M), envelop (E), and nucleoprotein (N). As an RNA virus, the coronavirus is genetically unstable and can easily mutate to escape host immune surveillance and clearance. Therefore, to prepare SARS-related vaccines, it is necessary to find antigens in the SARS-related coronavirus that have good genetic stability and immune response function.

Currently, inactivated virus particles are seldom used for vaccination because the virus particles include full genome of a virus, and the safety of such use raises concerns. Although conventional coronavirus may be easily obtained from cell culture, the unusual pathogenicity of SARS-related coronavirus and their uncertain genetic backgrounds make it impractical to prepare SARS-related coronavirus particles on large scales. The advancement of genetic engineering technology greatly facilitates the development of vaccines using sub-viral particle units, which are easier to manipulate and safer. If antigenic determinants with good antigenicity can be identified, using genetic engineering technology, one can conveniently reconstruct the antigen determinants to increase their genetic stability, antigenicity, and bio-safety. Therefore, it is clear that gene engineering technology can be conveniently used to prepare efficient vaccines using sub-viral particle units.

According to current research, among the four known structural proteins of the coronavirus, spike S is the one that can induce protective immune reactions. Some researchers have shown that the antigenic determinants of the spike S protein of the coronavirus are located at its C-terminus. Meanwhile, SARS-related coronavirus is known to cause infection in the respiratory tracts. However, we still do not have vaccines to prevent SARS.

In addition, it is known that the adenovirus itself can easily infect the mucous epithelials of the respiratory tracts, and induce immune reaction in the mucous epithelials of the respiratory tracts. Furthermore, immunogens carried on an adenovirus vector can be expressed, modified, folded, and presented in host cells, maintaining natural conformations of the immunogens to afford good biological activities.

The above described provides solid theoretical basis for researching and producing adenovirus SARS vaccines using defective adenovirus as a vector.

### SUMMARY OF THE INVENTION

The first objective of this invention relates to supplying an adenovirus SARS vaccine that can guard against the epidemics of atypical pneumonia and better prevent the occurrence and transmission of atypical pneumonia. Another objective relates to methods for preparing SARS vaccines using a defective adenovirus as a vector in combination with gene cloning and recombination techniques to produce adenoviral SARS vaccines. This invention also discloses the use of S gene of SARS-related coronavirus in vaccine preparation.

The objectives of this invention are realized as follows: by genetic engineering, the S gene of SARS-related coronavirus (the S gene is one of four structural genes in coronavirus, see details below) is combined with a defective adenovirus to construct a vaccine that can induce immunity of mucous membranes.

The sequences of the Spike S gene fragments used in the construction are as follows:

With the S gene sequence in GeneBank (GeneBank sequence No. gb AY278554.2) as a template, PCR primers are designed according to the S gene sequence as follows:

| | |
|---|---|
| V1 GGTCTAGAGT TGTGGTTTCA AGTGAT | [SEQ ID: 2] |
| V2 TTTCTAGACC ATGGGTTGTG TCCTTGCT | [SEQ ID: 3] |
| V3 TTTCTAGACC ATGGCATATA GGTTCAATG | [SEQ ID: 4] |
| V4 TAGGTACCAA TGCCAGTAGT GGTG | [SEQ ID: 5] |
| V5 TTGGTACCTC CGCCTCGACT TT | [SEQ ID: 6] |
| V6 CCGGTACCAT AAGTTCGTTT ATGTGT | [SEQ ID: 7] |

Among these primers, V1 and V4 form a pair for the amplification of the N-terminal fragment of the S gene; V2 and V5 form a pair for the amplification of the middle (M) region of the S gene; and V3 and V6 form a pair for the amplification of the C-terminal fragment of the S gene. (see FIG. 1).

### Preparation of Adenovirus SARS Vaccine:

First, collect and extract serum samples from patients who had recovered from the disease. Total RNA of coronavirus were obtained from the serum by separation and extraction. Then, the S gene is obtained by reverse transcription, sequencing, screening, and so on. Next, the S gene is cloned into a pShuttle plasmid to provide the desired clones (Deposit Institution: The Conservation Center for Typical Cultures in China; deposit date is May 18, 2003; deposit Nos. are CCTCC M 203036 E. coli DH5α/pShuttle-SN and CCTCC M 203070 E. coli DH5α/pShuttle-SC). These are then ligated with the adenovirus backbone plasmid pAdeno-X™ (Both pShuttle and pAdeno-X™ were purchased from CLONTECH Laboratory, Inc., U.S.A.). Then, they were used to co-transfect 293 cells. After further purification and confirmation, the 293 cells were expanded. The virus fraction was collected from the 293 cells. After separation and purification, this provides the SARS vaccine. The vaccine can be made into spray or other dosage forms. The main technical steps are illustrated in FIG. 4.

This invention relates to a genetically engineered vaccine, that is, a gene vaccine using a defective adenovirus as a vector. Th is vaccine makes use of an adenovirus vector, which readily infects mucous epithelials of respiratory tracts, to express protective antigenic proteins or peptides inside the mucous epithelials to induce immune responses in the respiratory tract mucous membranes. By inducing the immune responses in mucous membrane of respiratory tracts, the vaccine induces the production of corresponding antibodies in the subject to prevent virus invasion. As compared with the traditional inactivated virus particle vaccines, vaccines of this invention are safer and more convenient to use, without the restrain of muscle injection and other conditions.

At present, SARS is spreading quickly around the world. As a viral infectious disease, there is still no effective medicine to cure SARS. Under this condition, prevention is the best approach. It has been shown that the C-terminal of the spike S protein of the SARS-related coronavirus is where the antigen determinants are located. This invention is based on this finding. The gene encoding the spike S of the SARS-related coronavirus was synthesized, and then cloned into an adenovirus vector. A vaccine is produced after culture expansion, purification, and dosage preparation. The vaccine can effectively induce mucous membrane (epithelial cells) to produce antibodies and produce humoral immunity to prevent virus invasion. Thus, this vaccine can be widely used in clinic.

### BRIEF DESCRIPTION OF THE DRAWINGS

FIG. 1 shows a schematic for the PCR expansion of S gene fragments.

FIG. 2 shows results from restriction enzyme digestions of the recombinants of pShuttle-SC, pShuttle-SM, and pShuttle-SN.

FIG. 3 shows the sequencing results for the recombinants of pShuttle-SC, pShuttle-SM and pShuttle-SN.

FIG. 4 shows an outline of steps for vaccine preparation in accordance with one embodiment of the invention.

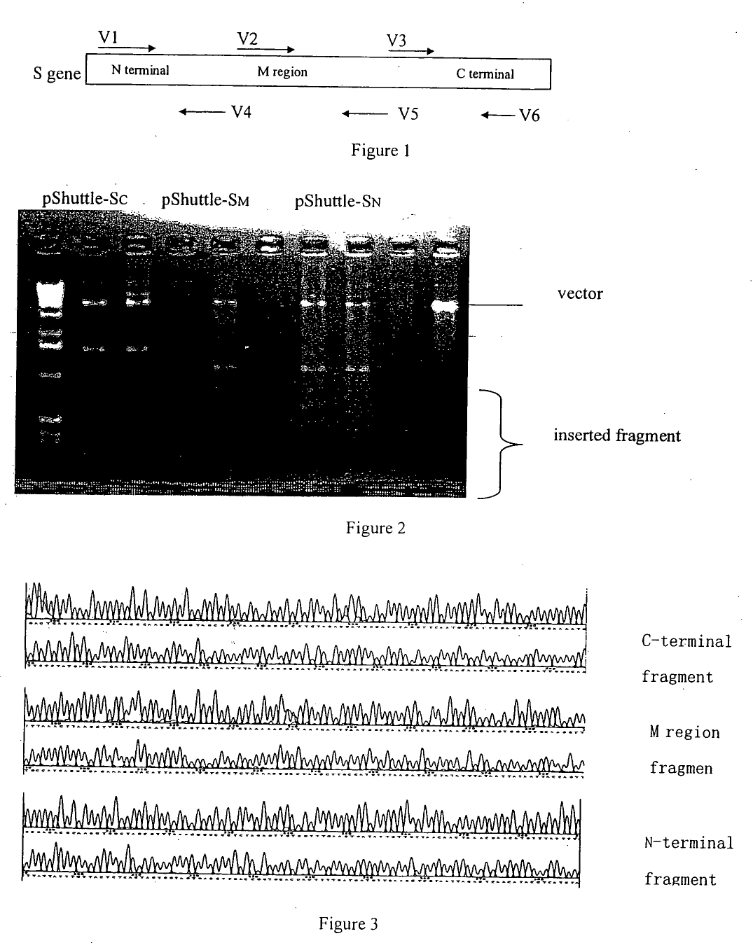

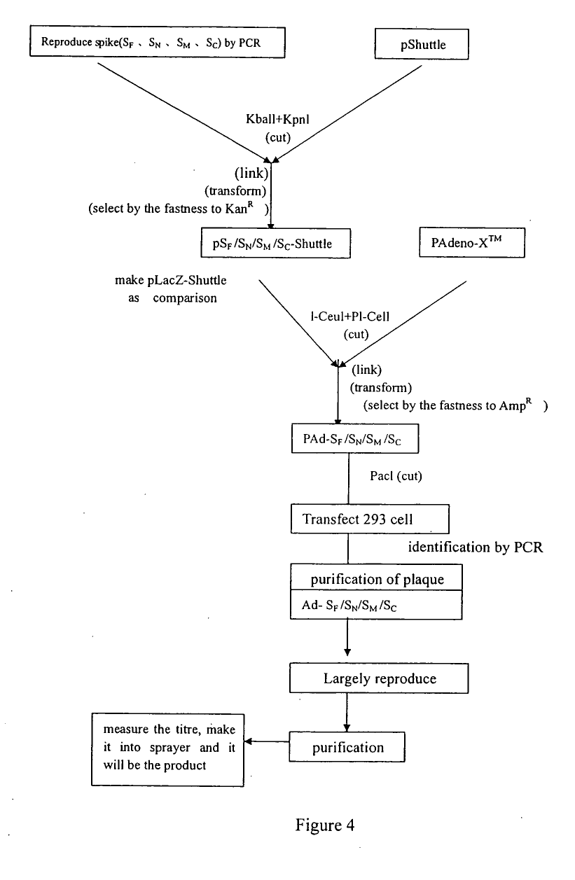

### DETAILED DESCRIPTION

The following describes practical examples to further illustrate embodiments of the invention.

### Example 1

### Preparation of Adenovirus Vector SARS Vaccine

The preparation of adenovirus vector SARS vaccine can be separated into two phrases: construction phase and expansion phase.

Construction Phase:

First, the genes (S_{F}, S_{N}, S_{M}, S_{C}) encoding the spike S protein of the SARS-related coronavirus are obtained and amplified by PCR. Then, these genes are digested with Xba1 and Kpn1 restriction enzymes at 37° C. Meanwhile, pShuttle is cut with the same restriction enzymes. The digested gene segments and the pShuttle plasmid are ligated. The recombinant plasmid is then transformed into E. coli DH5α, and positive clones (Kan^{R}) are screened and selected with Kanamycin. After culture expansion and purification, plasmids, pS_{F}/S_{N}/S_{M}/S_{C}-Shuttle are obtained. The plasmids, pS_{F}/S_{N}/S_{M}/S_{C}-Shuttle, and pAdeno-X™ are digested with 1-Ceu1 and P1-Sce1 restriction enzymes, and then ligated. The recombinant plasmids are transformed into E. coli DH5α and pAd-S_{F}/S_{N}/S_{M}/S_{C} are finally obtained by screening and selecting for positive clones (Amp⁺) that are resistant to Ampicillin.

Culture Expansion:

The pAd-S_{F}/S_{N}/S_{M}/S_{C} plasmids thus obtained are linearized with restriction enzyme Pac1, and transfected into packaging cells (packaging cells are from cell lines having the E1 gene of sub-group C, type 5 adenovirus (Ad5) integrated therein, such as the 293 cells). The Ad-S_{F}/S_{N}/S_{M}/S_{C} are identified by plaque screening/purification and PCR confirmation. Then, the 293 cells are transfected with Ad-S_{F}/S_{N}/S_{M}/S_{C} and grown in a large scale culture. The adenovirus-SARS particles produced from the 293 cells are collected by CsCl separation and purification. These may be made into a proper dosage form to afford the adenovirus-SARS vaccines. The dosage form may be a spray or an injection.

The SARS vaccines include the S gene of the SARS-related coronavirus and a defective adenovirus, which is a sub-group C, type 5 adenovirus, i.e., Ad5, without the E1 gene. The E3 gene region of this defective adenovirus may be completely lost, partially lost, or intact. The defective adenovirus includes a CMV promoter and a BGH polyA tail.

The sequence of the S gene of the SARS-related coronavirus cloned into the adenovirus vector may comprise the full length S gene.

The sequence of the S gene of the SARS-related coronavirus cloned into the adenovirus vector may comprise the S₁ domain sequence.

The sequence of the S gene of the SARS-related coronavirus cloned into the adenovirus vector may comprise the S₂ domain sequence.

The sequence of the S gene of the SARS-related coronavirus cloned into the adenovirus vector may comprise the S₁ and S₂ domain sequences (e.g., base Nos. 49˜3585 in SEQ ID NO: 1).

The sequence of the S gene of the SARS-related coronavirus cloned into the adenovirus vector may comprise the transmembrane region and a C-terminal fragment (e.g., base Nos. 3591-3654 in SEQ ID NO: 1).

### Example 2

The sequence of the S gene of the SARS-related coronavirus cloned into the adenovirus vector comprises the N-terminal segment. The other aspects are the same as in Example 1.

### Example 3

The sequence of the S gene of the SARS-related coronavirus cloned into the adenovirus vector comprises the middle (M) segment of the S gene. The other aspects are the same as in Example 1.

### Example 4

The sequence of the S gene of the SARS-related coronavirus cloned into the adenovirus vector comprises the C-terminal segment. The other aspects are the same as in Example 1.

### Example 5

The S gene recombinant pShuttle plasmid and characterization thereof:

The S gene and pShuttle are digested with restriction enzymes Xba1 and Kpn1 at 37° C. in a water bath. The digested fragments and the plasmid are ligated and transfected into E. coli DH5α. The cells are grown in a culture and screened for kanamycin resistant (Kan^{R}) positive clones to afford the pShuttle-SC, pShuttle-SM and pShuttle-SN, respectively. These plasmids are characterized by agarose gel electrophoresis and sequencing. Results are shown in FIGS. 2 and 3.

### Example 6

### Prevention of Green Monkey Kidney Cells (Vero E6) from SARS Attack

1. Incubate the Vero E6 cells on a 96-well plate at 2×10⁴ cells per well

2. After 24 hour culture, the cells are inoculated with adenovirus SARS vaccine. The inoculation is carried out as follows: (i) dilute the original virus stock 4 folds with culture medium 1640; (ii) after removing the culture medium from individual wells of the 96-well plate and washing the wells with PBS, add different dilutions of virus solutions into individual wells. Each dilution is added into 5 wells, 50 μL/well, with the culture medium 1640 (without virus) used as a negative control; (iii) incubate the plate at 37° C. with 5% CO₂ and saturated humidity for an hour; (iv) add culture medium 1640 containing 5% calf serum to the wells at 200 μL/well; and (v) incubate the plate at 37° C. with 5% CO₂ and saturated humidity.

3. After 24 hours, inoculate the cells with SARS virus as follows: (i) dilute SARS virus to 100TCID50 with culture medium 1640; (ii) after removing culture medium from the wells of the 96-well plate and washing with PBS, add different dilutions of SARS virus solution into wells. Each dilution is added to 5 wells, 50 μL/well, with the culture medium 1640 (without virus) used as a negative control; (iii) incubate the plate at 37° C. with 5% CO₂ and saturated humidity for an hour; (iv) add the culture medium 1640 containing 5% calf serum, 200 μL/well; and (v) incubate the plate at 37° C. with 5% CO₂ and saturated humidity.

4. After this, observe and record pathological changes of the cells every 12 to 24 hours. When calculating the results, the scores of pathological changes for all wells having the same dilution are summed to provide a pathological change index for that dilution. The results (expressed as % changes) from each group (control, SARS virus, or vaccine protected) are averaged and shown in the following table:

| | pathological | | | |
|---|---|---|---|---|
| | changes (%) | | | |
| group | 12 hours | 24 hours | 48 hours | 72 hours |
| Negative comparison | 0 | 0 | 0 | 0 |
| (1640 solution) | | | | |
| SARS virus attacking | 20 | 35 | 85 | 100 |
| group | | | | |
| Vaccine protective group | 5 | 10 | 25 | 40 |

What is claimed is:

1. An SARS vaccine, characterized in that the SARS vaccine comprises a sequence from the S gene of the SARS-related coronavirus and a replication-defective adenovirus.

2. The SARS vaccine according to claim 1, characterized in that said defective adenovirus is a sub-group C, type 5 adenovirus with a complete deletion in the E1 and E3 regions.

3. The SARS vaccine according to claim 1, characterized in that said defective adenovirus is a sub-group C, type 5 adenovirus with a complete deletion in the E3 region.

4. The SARS vaccine according to claim 1, characterized in that said defective adenovirus is a sub-group C, type 5 adenovirus with a partial deletion in the E3 region.

5. The SARS vaccine according to claim 1, characterized in that said defective adenovirus is a sub-group C, type 5 adenovirus with a deletion in the E1 region.

6. The SARS vaccine according to claim 1, characterized in that said defective adenovirus includes a CMV promoter and BGH polyA.

7. The SARS vaccine according to claim 1, characterized in that said SARS vaccine includes a full length S gene of the SARS-related coronavirus.

8. The SARS vaccine according to claim 1, characterized in that the sequence from the S gene includes the S₁ domain of the S gene of the SARS-related coronavirus.

9. The SARS vaccine according to claim 1, characterized in that the sequence from the S gene includes the S₂ domain of the S gene of the SARS-related coronavirus.

10. The SARS vaccine according to claim 1, characterized in that the sequence from the S gene includes the S₁ and S₂ domains of the S gene of the SARS-related coronavirus.

11. The SARS vaccine according to claim 1, characterized in that the sequence from the S gene includes the transmembrane domain and a C-terminal fragment of the S gene of the SARS-related coronavirus.

12. A method for preparing an SARS vaccine, comprising:
(1) obtaining S gene of the SARS-related coronavirus;
(2) recombining the S gene and a defective type adenovirus to produce a recombinant adenovirus;
(3) transfecting packaging cells with the recombinant adenovirus;
(4) expanding the transfected packaging cells, and separating and purifying recombinant viral particles produced from the packaging cells for making the SARS vaccine in a selected dosage form.

13. The method according to claim 12, characterized in that the obtained S gene is cloned into a pShuttle plasmid and then ligated with an adenovirus plasmid pAdeno-X™.

14. The method according to claim 12, characterized in that the obtaining the S gene is by polymerase chain reaction (PCR) using PCR primers based on the sequence of the S gene, wherein the PCR primers are as follows,

| | |
|---|---|
| V1 GGTCTAGAGT TGTGGTTTCA AGTGAT | [SEQ ID: 2] |
| V2 TTTCTAGACC ATGGGTTGTG TCCTTGCT | [SEQ ID: 3] |
| V3 TTTCTAGACC ATGGCATATA GGTTCAATG | [SEQ ID: 4] |
| V4 TAGGTACCAA TGCCAGTAGT GGTG | [SEQ ID: 5] |
| V5 TTGGTACCTC CGCCTCGACT TT | [SEQ ID: 6] |
| V6 CCGGTACCAT AAGTTCGTTT ATGTGT | [SEQ ID: 7] |

15. The method according to claim 12, characterized in that the packaging cells have the E1 gene of sub-group C, type 5 adenovirus (Ad5) integrated therein.

16. The method according to claim 15, characterized in that the packaging cells are 293 cells.

17. The method according to claim 12, characterized in that the selected dosage form is a spray or an injection.

18. A SARS vaccine comprising a sequence from the S gene of the SARS-related coronavirus.
